(19) United States
(12) Patent Application Publication
(10) Pub. No.: US 2016/0313292 A1
(43) Pub. Date: Oct. 27, 2016

(54) METHOD AND APPARATUS FOR MEASURING CHARACTERISTICS OF A HEATED FLUID IN A HOSTILE ENVIRONMENT

(71) Applicant: Petroleum Analyzer Company, LP, Houston, TX (US)

(72) Inventors: Chetan Desai, Sugar Land, TX (US); Patrick Dalbello, Houston, TX (US); David Anthony Malaguti, Stoneham, MA (US); Jonathan Stephen Lilley, Woburn, MA (US); Daniel Airey, Woburn, MA (US)

(73) Assignee: Petroleum Analyzer Company, LP, Houston, TX (US)

(21) Appl. No.: 15/061,602

(22) Filed: Mar. 4, 2016

Related U.S. Application Data
(60) Provisional application No. 62/152,199, filed on Apr. 24, 2015.

(51) Int. Cl. G01N 33/28; G01N 11/02; G01N 1/38; G01N 9/32

(52) U.S. Cl. CPC G01N 33/28; G01N 9/32; G01N 11/02; G01N 1/38

(57) ABSTRACT

In the production of petroleum-based products in a refinery, viscosity and density are accurately and repeatedly measured at an elevated temperature, sometimes in a hostile environment. A densitometer and viscometer are located in a non-purged enclosure, but the electrical controls that could cause a spark to ignite fumes are located in a purged enclosure with connections there between. A heat pipe extending from the purged enclosure to the non-purged enclosure accurately controls the temperature of a sample during testing. For difficult to handle petroleum-based products such as asphalt, a sample conditioning system prepares the sample prior to testing.

### CROSS-REFERENCE TO RELATED APPLICATIONS

This utility non-provisional application claims priority to U.S. Provisional Application No. 62/152,199, filed on Apr. 24, 2015.

### BACKGROUND OF THE INVENTION

1. Field of the Invention

The present invention relates to measuring the characteristics of heated fluids during the production process and, more particularly, to measuring the characteristics of petroleum-based products during production, which can occur in a hostile environment.

2. Description of the Prior Art

There are more than seven hundred petroleum refineries worldwide with a capacity to produce in excess of eighty million barrels of oil per day. These refineries operate in virtually every country of any size on earth. In the United States, it is estimated there are one-hundred and thirty-seven refineries having an estimated capacity of approximately seventeen million barrels of oil per day. The demand for energy continues to rise along with pressures on producers to streamline and speed up production, plus increase yield and operate more efficiently.

A typical barrel of oil yields approximately fifty percent (50%) gasoline, fifteen percent (15%) fuel oil, twelve percent (12%) jet fuel, with the remainder being made into diesel fuel, asphalt, lubrication oil and other refined products, but these actual percentages vary drastically with refineries and the source of the crude oil. Viscosity is one of the most critical measurements of product quality for virtually every petroleum refinery product. Improvements in viscosity measurement enable petroleum refineries to make significant improvements in product quality, costs and output.

Asphalt is one of the more difficult petroleum products to manufacture because the asphalt has to be maintained at a high temperature during production. Roads on which the asphalt is used have radically different environments throughout the world and at different times of the year. All customers for asphalt have asphalt pavement specifications that are suitable for their region.

The raw material for making asphalt is basically what is left at the bottom of the barrel of crude oil when all of the higher value materials have been extracted and refined. That material at the bottom of the barrel is very non-homogeneous, and can vary radically in make-up from barrel-to-barrel, depending on the source of the crude. Variations in the refinery process conditions can also have an impact on the asphalt.

Customer specifications are based on international standard test methods that normally utilize standard laboratory test equipment. These laboratory tests are done periodically throughout production, and the process is adjusted based upon the test results. The petroleum based product may be tested in a storage tank, and if necessary re-blended to meet the customer specifications. Unfortunately, asphalt characteristics can vary significantly between lab tests.

Refineries utilize in-line measurements to enhance consistency of the petroleum-based products that are produced. In-line viscosity and density measurements enhance consistency of the product produced. Some petroleum-based products are graded and sold based upon their viscosity characteristics. Viscosity is the measurement of a fluid's resistance to flow. Highly precise viscosity measurements are required for some petroleum-based products.

Just some of the tests that are made on petroleum-based products are as follows:

| ASTM # | TITLE |
|---|---|
| D 7483-8 | Standard Test Method for Determination of Dynamic Viscos- |
| | ity and Derived Kinematic Viscosity of Fluids by Oscillating |
| | Viscometer |
| D 3381-05 | Standard Specification for Viscosity-Graded Asphalt Cement |
| | for Use in Pavement Construction |
| D 2170-07 | Standard Test for Kinematic Viscosity of Asphalt (Ditumens) |
| D 445-06 | Standard Test Method for Kinematic Viscosity of Transparent |
| | and Opaque Fluids (in Calculation of Dynamic Viscosity) |
| D 341-09 | Standard Practice for Viscosity-Temperature Charts for Liquid |
| | Petroleum Products |
| D 2171-07 | Standard Test Method for Viscosity of Asphalts by Vacuum |
| | Capillary Viscometer |

Because asphalt is normally hard at room temperature, viscosity of asphalt is normally measured at 135° C., according to ASTM standard tests. In performing the ASTM standard test, it is very important that the product being tested be maintained at a very accurate temperature.

### SUMMARY OF THE INVENTION

It is an object of the present invention to provide an analyzer that will accurately measure the viscosity of a product during production.

It is another object of the present invention to provide a viscosity measuring analyzer that can be used in a hostile environment that may be explosive.

It is still another object of the present invention to provide an accurate measurement of viscosity and density of petroleum-based products during production.

It is yet another object of the present invention to provide an analyzer that has an accurately controlled heat pipe extending from a sealed electrical control enclosure into an unsealed enclosure where the sample is heated (or cooled) to a desired temperature for measurement.

It is still another object of the present invention to utilize a heat pipe to accurately control the temperature of a sample being tested, which heat pipe extends from a sealed electrical enclosure to an unsealed enclosure that is subject to a hostile, and possibly explosive, work environment to accurately control the temperature of the sample being tested.

It is yet another object of the present invention to precondition certain samples prior to feeding the samples into the analyzer.

It is still another object of the present invention to precondition samples, such as asphalt, to an approximate temperature desired before feeding the preconditioned sample to an analyzer for subsequent determination of viscosity and/or density.

It is still a further object of the present invention to take samples from a continuous flow process, which samples are then accurately measured for viscosity and/or density.

Because the analyzer is designed for use in a hostile (possibly explosive) environment, all the electronics are contained in a purged enclosure for electronics, including a computer (programmable logic controller), processor, solenoid valve contacts and controls for a heat pipe.

In a non-purged enclosure a densitometer receives a sample where the density is measured followed by a viscometer measuring the viscosity. At the time of the measurement, a heat pipe extends from the purged enclosure to the non-purged enclosure to accurately control the temperature of the sample located inside of the viscometer. Low voltage/current sensors feed the signals from the non-purged enclosure to the purged enclosure. This allows a very accurate measurement of density and viscosity at a tightly controlled temperature.

For some samples, such as asphalt, the sample may have to be preconditioned prior to feeding into the purged enclosure for measurement with a densitometer and/or viscometer. A sample conditioning system may have a heat exchanger to set the temperature of the sample within a general range close to the desired measured temperature. Thereafter, the conditioned sample is fed to the non-purged enclosure where the temperature is adjusted further by the heat pipe at the time of measuring in the viscometer to get a very accurate temperature at the time of the viscosity measurement.

Depending upon the work environment, the purged enclosure may need to have some type of temperature control such as a Vortex cooler system. Also, a purging system for the purged enclosure may need to be utilized. By appropriate touch screen display, an end user can see exactly what is occurring during the measurement of samples taken from the continuous manufacturing process. Samples are continuously obtained and measurements made to make sure the product being produced remains within specification.

### DESCRIPTION OF THE DRAWINGS

FIG. 1 is a simplified block diagram of an analyzer with a sample conditioning system.

FIG. 2 is a simplified block diagram of a viscometer with a sample conditioning system for use in a hostile environment.

FIG. 3 is a detailed front view of the viscometer shown in the simplified block diagram of FIG. 2.

FIG. 4 is an exploded perspective view of the contents of the left side of

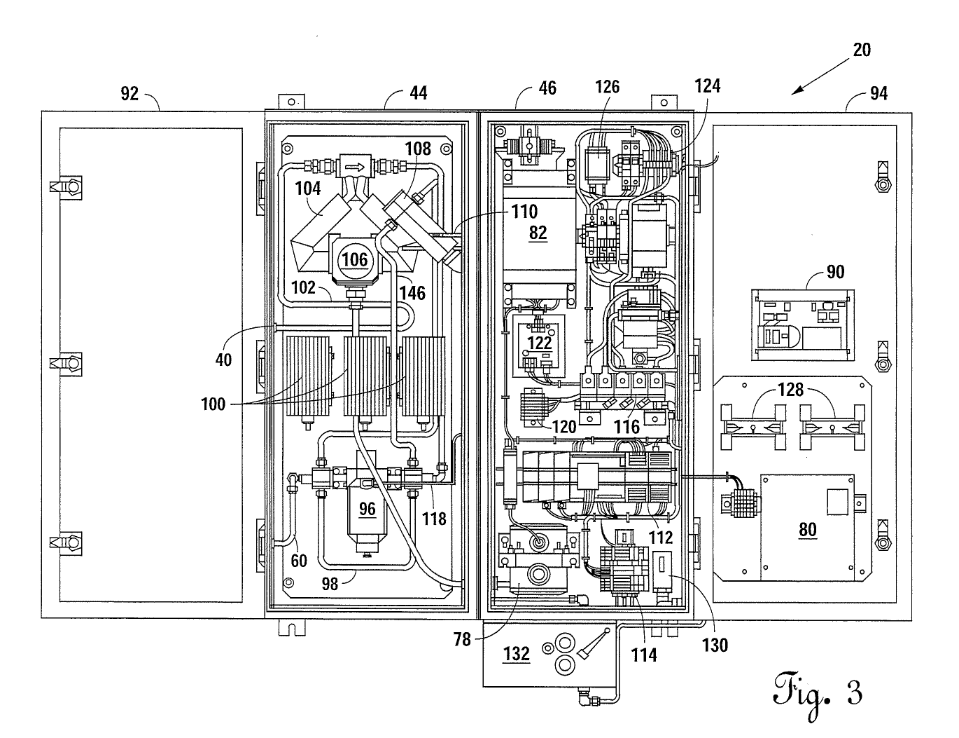

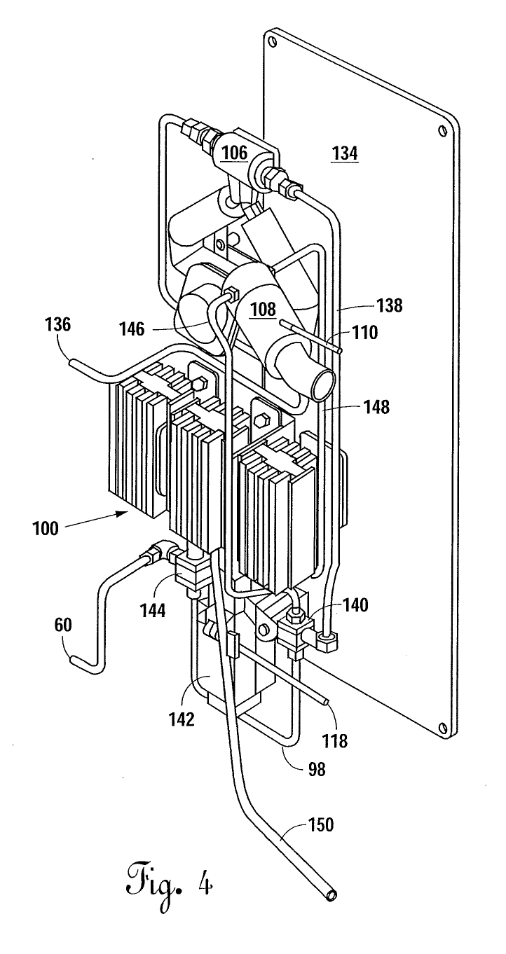

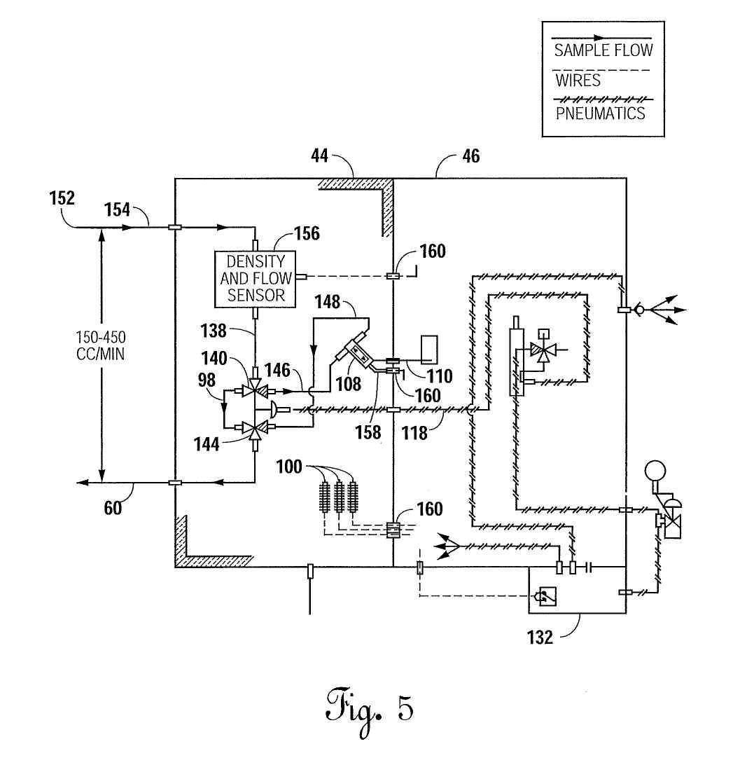

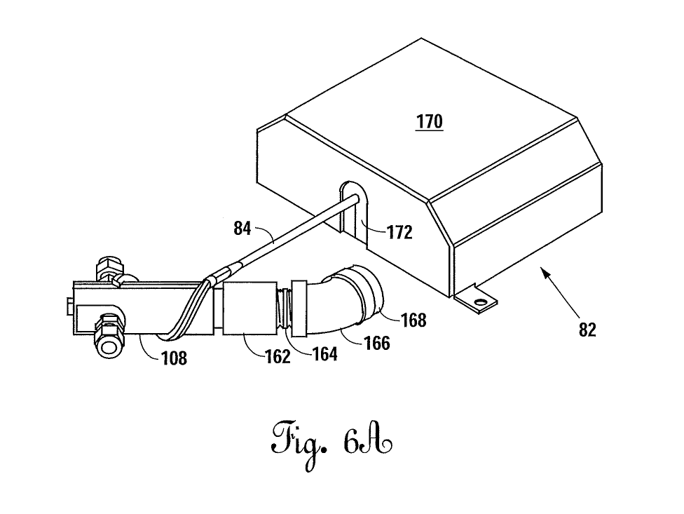

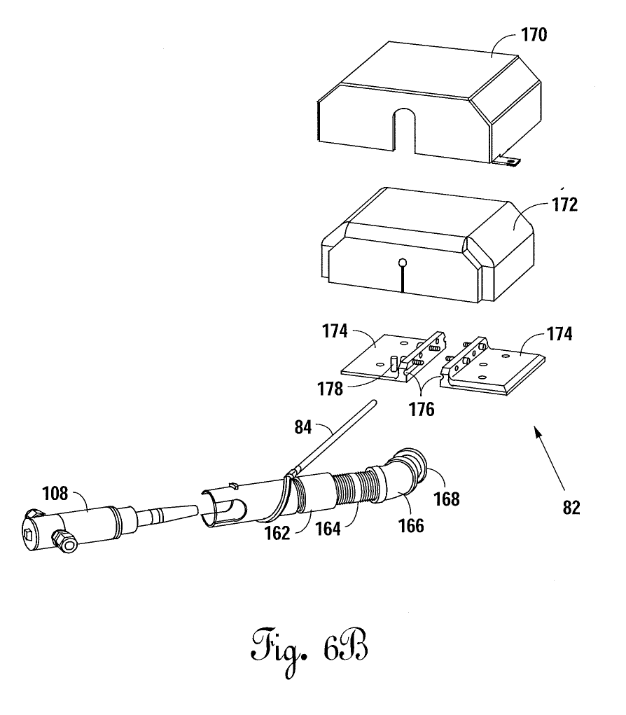

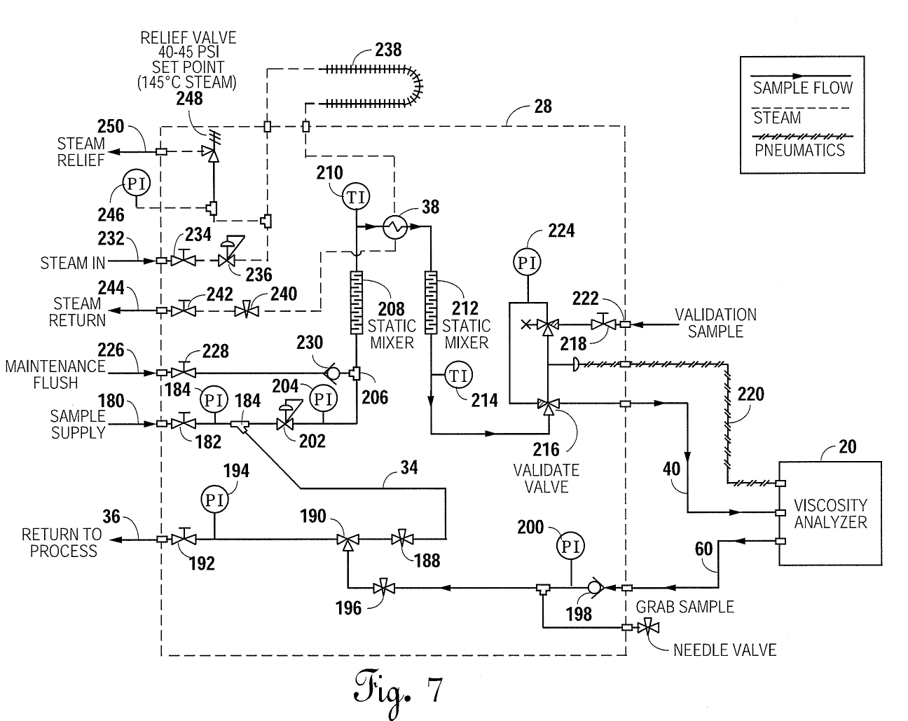

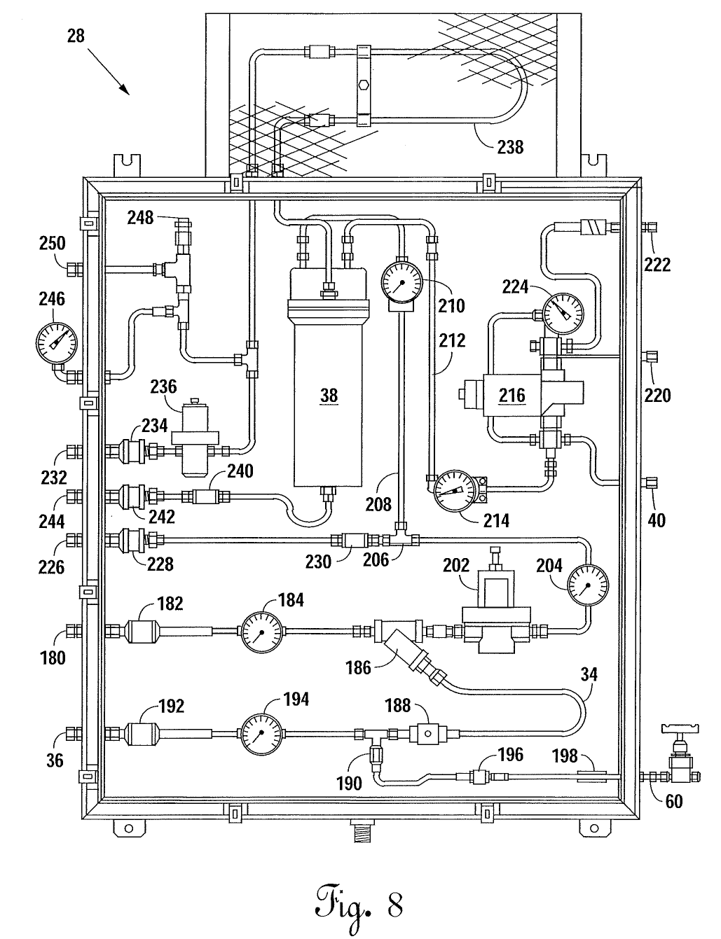

FIG. 3.

FIG. 5 is a simplified flow diagram in the left side shown in FIG. 3.

FIG. 6A is a perspective view of the heat pipe used in FIGS. 3, 4 and 5.

FIG. 6B is an exploded perspective view of a heat pipe shown in FIG. 6A.

FIG. 7 is a simplified flow diagram of a sample conditioning system that connects to the viscosity analyzer shown in FIG. 3.

FIG. 8 is a front view of a sample conditioning system illustrated schematically in FIG. 7.

FIG. 9 is an optional Vortex cooler that may be used in the purged enclosure for electronics shown on the right side of FIG. 3.

FIG. 10 are measurements taken of the flow rate, viscosity and temperature of the viscometer as shown in FIG. 3 with the sample conditioning system as shown in FIG. 8.

### DESCRIPTION OF THE PREFERRED EMBODIMENT

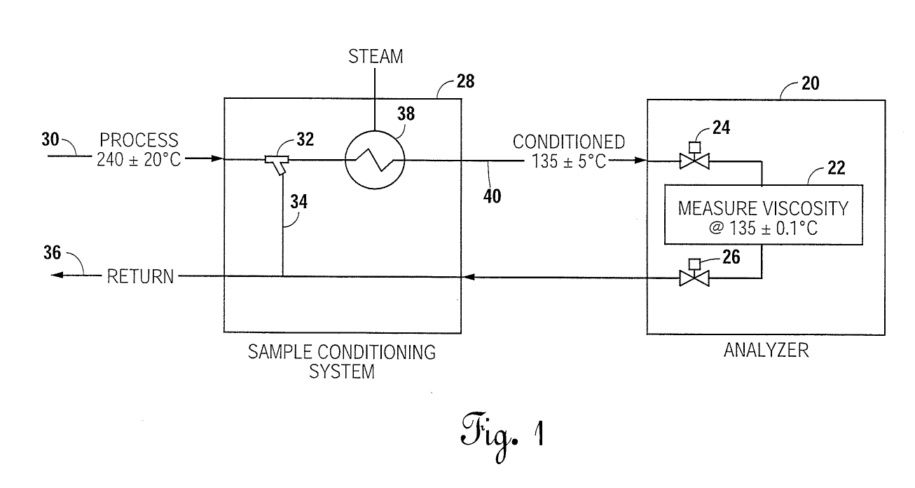

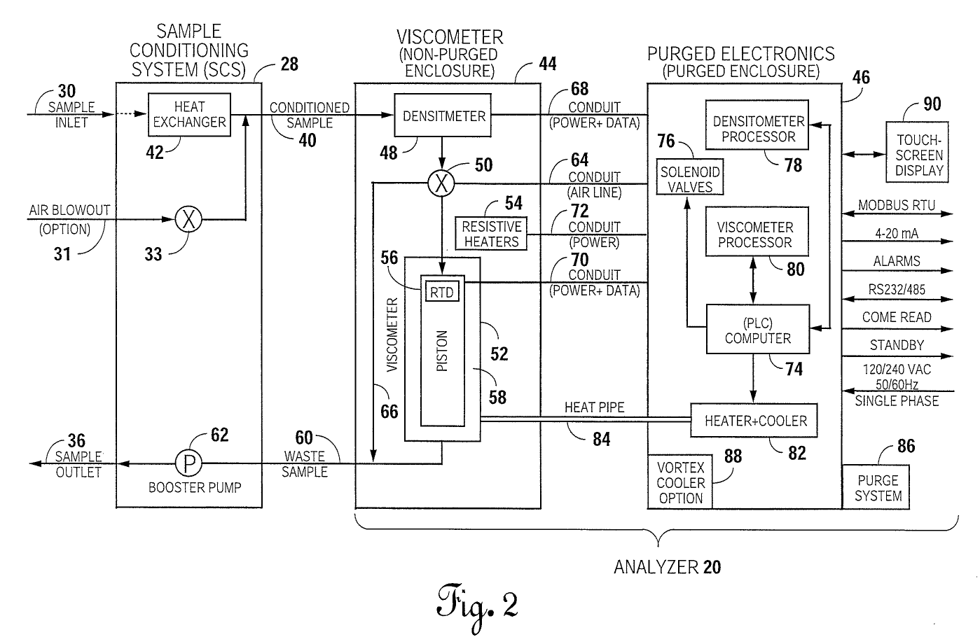

Referring to FIG. 1, a simplified block diagram is shown of an analyzer 20 that has a viscometer 22 therein to measure viscosity. If the viscometer 22 is measuring asphalt, it would be at 135° C. ±0.1° C. Shut-off valves 24 and 26 can stop the flow through the viscometer 22.

If the analyzer 20 is analyzing a sample such as asphalt, a sample conditioning system 28 will have to be used. In the sample conditioning system 28, asphalt will flow in through the process line 30 at approximately 240° C. within a range of ±20° C. Hot asphalt will continuously flow in through the process line 30, sampler 32, bypass line 34 and out return line 36. This ensures that the same asphalt that is being produced is what will be sampled inside of sample conditioning system 28. A sample of what is being processed is selected inside of sampler 32 and fed through steam heat exchanger 38 to lower the temperature of the sample selected to approximately 135° C. within a ±5° C. range, which selected sample is fed through the conditioned sample line 40 into the analyzer 20. The conditioned sample flows through shut-off valve 24 into the viscometer 22 where the viscosity is measured before the sample returns through shut-off valve 26 and out return line 36.

Prior to measurement, the sample conditioning system 28 cools the selected sample with steam and prevents wide variations in the sample temperature. Also, the sample conditioning system 28 ensures that a homogeneous sample with the particles removed is delivered to the analyzer 20. With the sample conditioning system 28 being used, ASTM Standard D7483 can now be used to measure the viscosity of the selected samples.

Referring now to FIG. 2, the sample conditioning system 28 and the analyzer 20 are shown in a more detailed pictorial block diagram. The sample inlet still provides a sample through process line 30. The sample will flow through a heat exchanger 42 (which can be steam heat exchanger 38 shown in FIG. 1) to provide a conditioned sample through conditioned sample line 40 to the analyzer 20. The analyzer 20 has a non-purged enclosure 44 and a purged enclosure 46. Inside of the non-purged enclosure 44 the sample is received through a densitometer prior to feeding through valve 50 into the viscometer 52. The non-purged enclosure 44 may be heated by resistive heaters 54 so that the non-purged enclosure 44 is at an elevated temperature. The viscometer 52 may have a piston 56 and a temperature control sensor jacket 58.

After measurement of the sample in the densitometer 48 and the viscometer 52, the sample waste returns through the sample waste line 60 to return line 36. Booster pump 62 may be necessary to create a vacuum to draw the used sample through the sample waste line 60. Between measurements inside of the viscometer 52, valve 50 can be operated via airline 64 to continuously flow sample material via bypass line 66 out waste sample line 60 and return line 36. Power and data are provided to/from the densitometer 48 and the viscometer 52 via power/data conduits through seal-off glands 68 and 70. The resistive heaters 54 receive their power through power conduit 72.

Referring now to the purged enclosure 46, everything is controlled by a computer 74, also called a programmable logic controller (PLC). The computer 74 operates the solenoid valves 76, which in turn operates valve 50 via air line 64. Also, the computer 74 operates the densitometer processor 78. Also, the computer 74 operates the viscometer processor 80. Further, the computer 74 operates the heater/cooler 82 provided by heat pipe 84. The purged enclosure 46 also has a purge system 86. Optionally, the purged enclosure 46 may have a Vortex cooler 88.

Because the purged enclosure 46 may have sparks therein that could ignite fumes in a hostile environment, purged enclosure 46 has to stay purged. To ensure this occurs, the connections between non-purged enclosure 44 and purged enclosure 46 all use seal-off glands there between. Also, the data being received from the densitometer 48 and/or viscometer 52 is of very low power that would not ignite any flammable gases that may be contained inside of non-purged enclosure 44.

On the front of the purged enclosure 46 is located a touch screen 90 to allow an external interface with the analyzer 20. Normal power and alarms will be provided to the purged enclosure 46, but through seal-off glands (not shown) to maintain the purged atmosphere inside purged enclosure 46.

If the sample conditioning system 28 and/or the analyzer 20 need to be purged, a blast of air can be provided via air blowout line 31 and valve 33 to analyzer 20 on conditioned sample line 40.

Referring now to FIG. 3, analyzer 20 is shown in more detail. The non-purged enclosure 44 is shown on the left with the purged enclosure 46 being shown on the right. The non-purged lid 92 for enclosing the non-purged enclosure 44 is shown on the left and the purged lid 94 for the purged enclosure 46 is shown on the right.

The non-purged enclosure 44 is an oven section with a shut-off valve 96 located at the bottom thereof. Below the shut-off valve 96 is a continuous bypass loop 98 that ensures the analyzer 20 is always measuring the latest and freshest sample. Approximately every five minutes, the shut-off valve 96 is actuated so that the valves are momentarily changed to a different direction of flow so that a sample can be taken and a measurement made.

Immediately above the shut-off valve 96 are heaters 100. Heaters 100 are positive temperature coefficient heaters that are self-limiting. These heaters 100, due to their self-limiting features, ensure that the surface temperature of the heaters never exceeds their certified “T-rating” (e.g. T3 heaters will have a maximum surface temperature of no more than 200° C.). This ensures that the non-purged enclosure 44 can have a “T rating” to operate in a hazardous zone. An example of such a self-limiting positive temperature coefficient heater is made by Intertec.

The condition sample line 40 connects through U-shaped tubing 102 to preheat the sample before entering the triangular loop 104 at the top of the densitometer 106. The densitometer 106 measures the density of the fluid flowing in through the condition supply line 40. The densitometer 106 provides (a) the density, (b) flow rate and (c) temperature of the sample. These three physical parameters are measured before the sample enters the viscosity sensor 108, also known as viscometer. A sample is trapped inside of the viscometer 108 by momentary actuation of the shut-off valve 96. After the temperature inside of the viscometer 108 is stabilized by heat pipe 110, which heat pipe 110 extends from the purged enclosure 46 into the non-purged enclosure 44, the viscosity is measured. The heat pipe 110 provides a very precise temperature to the sample being analyzed in the viscometer 108.

Inside of the purged enclosure 46 is a computer 112 also known as a programmable logical controller (PLC). The computer 112 initiates the functions of the analyzer based upon user inputs through the touch screen 90 that is on the purge lid 94. The computer 112, after the set points have been programmed in, initiates the analyzer 20 to start the density and viscosity measurements. The computer 112 receives inputs from the densitometer processor 78 to control operation of the densitometer 106. Terminal block 114 provides for external input/output connections where the final values requested by the customer can be set into the computer 112. Through the terminal block 114, the customer can tell if the alarms are set and the temperature in which the viscosity is measured, or any other information of interest to the customer that may be used to control the analyzer 20. Above the computer 112 are solenoid valves 116 which actuate a pneumatic valve inside of the non-purged enclosure previously referred to as shut-off valve 96. Pressurized air is provided to shut-off valve 96 via air pressure line 118, which is controlled by solenoid valve 116 inside of the purged enclosure 46. While not in use in the present design, additional solenoid valves 116 are provided for further expansion or requirements of the customer. These additional solenoid valves 116 may be operated through terminal block 120.

The heater/cooler 82 is a Peltier type heater/cooler that is controlled by an H-bridge 122 located there below. The H-bridge 122 powers the Peltier heater/cooler 82. With a Peltier heater/cooler having an H-bridge 122, the polarity of the Peltier heater/cooler can be reversed resulting in either heating or cooling, depending upon the direction the voltage is applied by the H-bridge 122. The Peltier heater/cooler 82 provides either heat or cooling to the heat pipe 110 that extends from the purged enclosure 46 to the non-purged enclosure 44. The operation of the Peltier heater/cooler 82 in conjunction with the heat pipe 110 will be explained in more detail subsequently in connection with FIGS. 6A and 6B. Good control of the Peltier heater/cooler 82 means good control of the heat pipe 112 and good control of the temperature at the time of the viscosity measurement.

Power to the analyzer 20 comes in through power supply breakers 124. The different voltage levels are provided as needed for the analyzer 20. An EMI filter 126 eliminates electromagnetic interference being received through the power supply.

Isolation barriers are used on any electrical wires that may go from the purged enclosure, including to the non-purged enclosure. This is important to make sure that any spark that might originate in the electrical portion contained in the purged enclosure 46 (which is isolated) does not make it to the non-purged enclosure 44, which is in a hostile environment. Positive pressure is maintained in purged enclosure 46 to keep hazardous fumes from coming in contact with the electrical portion contained in the purged enclosure 46. The purged enclosure 46 provides a safe zone inside of the analyzer 20. For connections between the purged enclosure 46 and the non-purged enclosure 44, seal-off glands are used between the two. The seal-off glands are certified according to safety agencies.

The touch screen 90 in purge lid 94 provides human-machine interface. Behind the touch screen 90 is a memory card that contains data as to what is to be continuously monitored. Fans 128 circulate the air inside of the purged enclosure 46 to keep the temperature therein basically uniform.

The viscometer processor 80, which connects to the computer 112, provides the controls for the viscometer 108. The viscometer processor 80 controls the action of the piston 56 inside of viscometer 52 (see FIG. 2). A typical viscometer that can be used is manufactured by Cambridge Viscosity, Inc.

If the analyzer 20 is being used in a hot environment, it may be necessary to provide cooling inside of the purged enclosure 46. A cooler that has been found particularly useful is a vortex cooler as will be explained in more detail subsequently to receive air there from and replace the removed air with pressurized air that is from a safe source, such as air lines in a processing facility. The purged system and vent 132 is provided at the bottom of the purged enclosure 46.

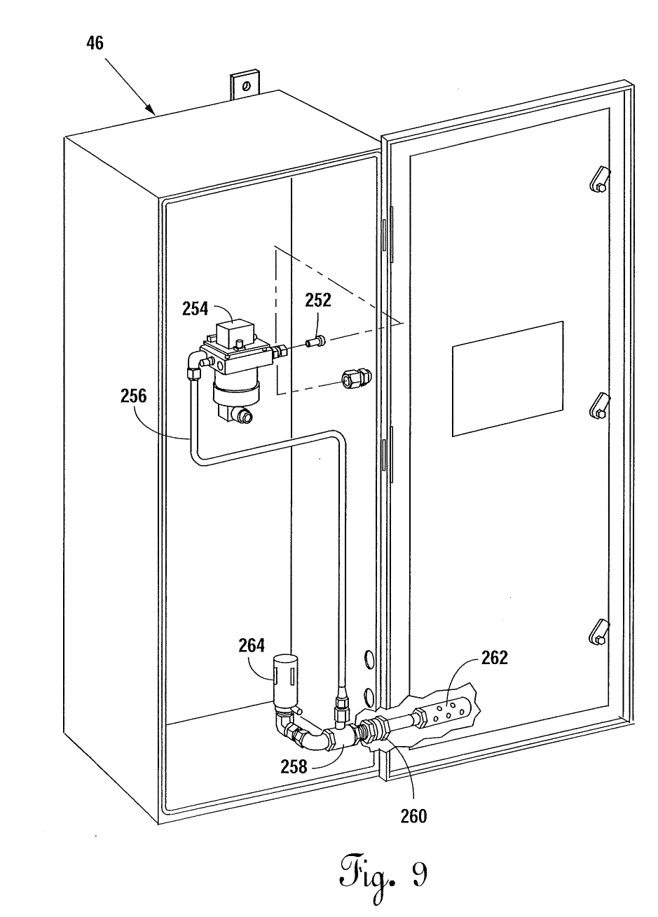

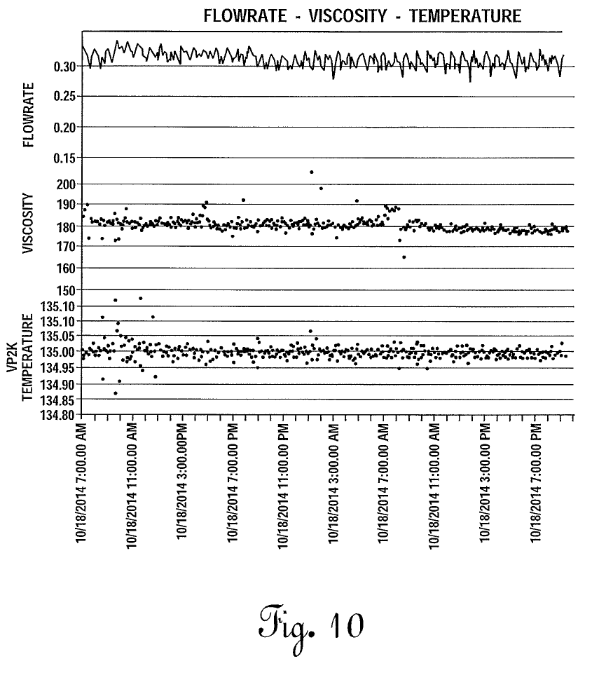

Referring now to FIG. 4 in combination with previous FIGS. 2 and 3, a partial exploded perspective view is shown of the internal components of the non-purged enclosure 44. Mounted on back panel 134 is the densitometer 106. A sample line 136 provides the input to the densitometer 106. Tube 138 connects the densitometer 106 to valve 140 operated by valve sub-assembly 142. Continuous bypass loop 98 allows the fluid continuously being sampled to bypass valve sub-assembly 142 and flow through valves 140 and 144 out sample waste line 60.

Connecting from valve 140 to the viscometer 108 is tube 146 and connecting back from the viscometer 108 to valve 144 is tube 148. Heaters 100 make sure the temperature inside of the non-purged enclosure 44 is maintained at a predetermined temperature. Test line 150 allows a sample of predetermined characteristics to be run through the analyzer 20 to make sure the analyzer 20 is operating properly and calibrated. Air pressure line 118 provides pressure to operate the valve sub-assembly 142.

Referring now to FIG. 5, a schematic flow diagram of what has been shown in more detail in FIGS. 3 and 4 is provided. While a pictorial flow diagram is shown for the non-purged enclosure 44, only some of the electronics are shown in the purged enclosure 46. The non-purged enclosure 44 is a heated enclosure that is insulated. A sample 152 is provided via sample input line 154 to a density/flow sensor 156. From the density/flow sensor 156, the sample flows through tube 138 to valve 140. Most of the time the sample 152 will flow through valve 140, continuous bypass loop 98 and out valve 144 to sample waste line 60. However, upon receiving pressurized air through air pressure line 118, valves 140 and 144 are switched so that the sample now flows through tube 146 to viscometer 108, whose temperature is accurately controlled by heat pipe 110. After measurement inside of the viscometer 108, the sample flows via tube 148 and valve 144 out sample waste line 60. Low voltage signals or data is provided through conduit 158 via seal-off gland 160 to purged enclosure 46. The electric heaters 100 heat the non-purged enclosure 44. Seal-off glands 160 are provided for each connection that goes between the non-purged enclosure 44 and the purged enclosure 46. The purged system and vent 132 are pictorially illustrated for the purged enclosure 46.

Referring now to FIGS. 6A and 6B in combination, the Peltier heater/cooler 82 and the heat pipe 84 will be explained in more detail. A viscometer 108 is shown which connects to heat pipe 84 via coupling 162. By the heat pipe 84 wrapping around the coupling 162 for the viscometer 108 ensures the sample being measured inside of the viscometer 108 is the same temperature as the temperature of the heat pipe 84. Electrical connections for the viscometer 108 are provided through coupling 162, nipple 164 and elbow 166 and through sensor mount bushing 168.

Inside of the Peltier cover 170 is located insulation 172. Contained inside of the insulation 172 are platen clamps 174. The platen clamps 174 each have a groove 176 therein that is designed to receive the end of the heat pipe 84. The clamping of the heat pipe 84 inside of the groove 176 of the platen clamps 174 ensures a good heat transfer there between. A temperature sensor 178 monitors the temperature of the platen clamps 174 and hence the heat pipe 84. This temperature is fed back to the computer 112 contained in purged enclosure 46 (see FIG. 3). The temperature sensor 178 is a normally closed (NC) temperature sensitive disc thermostat attached to the surface of the platen clamp 174. If the temperature of the platen clamp 174 exceeds a preset temperature limit, the temperature sensor 178 creates a circuit condition which removes power from the Peltier heater/cooler 82. When the temperature of the platen clamps 174 drops below the preset temperature limit, the normally closed switch of the temperature sensor 178 will close to restore power to the Peltier heater/cooler 82.

By wrapping the heat pipe 84 around the coupling 162, there is very good heat transfer to the viscometer 108. Temperature measurements from inside of the viscometer 108 are fed back through the inside of coupling 162, nipple 164, elbow 166 and sensor mount bushing 168 to the inside of the purged enclosure 46. From inside of the purged enclosure 46, the temperature of the Peltier heater/cooler 82 is very accurately controlled, which heats (or cools) the platen clamps 174. By use of the H-bridge 122 (see FIG. 3), the Peltier heater/cooler 82 can be switched from heating to cooling or vice-versa. This type of heating/cooling is sometimes referred to as a thermoelectric heater/cooler. A common brand is made by a company called TECA. The heat that is generated inside of the purged enclosure 46 is mechanically transferred through the heat pipe 84 to the viscometer 108 inside of the non-purged enclosure 44, which viscometer 108 may be in a hostile environment. No large currents are in the hostile environment of the non-purged enclosure 44.

The sample conditioning system 28 as previously pictorially shown in FIGS. 1 and 2 will be described in more detail in FIGS. 7 and 8. FIG. 7 is a pictorial illustration of the sample conditioning system 28, while FIG. 8 is a more detailed embodiment of the sample conditioning system 28Prov. There are some products that require more conditioning before being testing by the analyzer 20 than others. A typical example is asphalt, which has to be heated to a higher temperature during the manufacturing process. Exact standards have been set by the ASTM for measuring characteristics of asphalt, particularly viscosity and density. In most refineries, asphalt is processed at approximately 240° C. with a variation being ±20° C. However, ASTM standards require the viscosity of asphalt to be measured at 135° C. with a ±variation being within ±0.1° C. The object of the sample conditioning system 28 is to get the temperature of the asphalt to approximately 135° C. within +5° C. Thereafter, the conditioned sample can be fed to the analyzer through a conditioned sample line 40.

From the manufacturing process, a sample is continuously provided through sample supply line 180 into the sample conditioning system 28. After the sample flows through a control valve 182 and pressure measured in pressure gauge 184, the sample flows through “Y” strainer 186. The “Y” strainer 186 has a mesh strainer therein (not shown) with the sample continuously flowing through the large portion of the “Y” strainer 186, through bypass line 34, through needle valve 188, through a venturi tee eductor 190 and out control valve 192 to return line 36. Pressure of the sample is measured by pressure gauge 194 immediately prior to discharging from the sample conditioning system 28.

The “Y” strainer 186 is almost self-cleaning. The sample being selected for analysis comes from the center of the “Y” strainer 186, which generally does not have particles therein. After selecting the sample, the next slug of material that flows through the “Y” strainer 186 flushes away most of the particles that have been accumulated. The filter inside of the “Y” strainer 186 can operate a long time without having to be cleaned. The eductor 190, due to the flow of fluid there through, creates a vacuum that sucks product from the viscosity analyzer 20 through the needle valve 196 and check valve 198 via sample waste line 60. Also, another pressure gauge 200 may monitor pressure in the sample waste line 60. The vacuum created by the eductor 190 helps draw the sample through the analyzer 20.

Once the sample is selected from the “Y” strainer 186, the sample flows through pressure regulator 202 as measured by pressure gauge 204 to T-connector 206. From the T-connector 206, a sample flows through a static mixer 208. The static mixer 208 appears to be the same as a normal tube but inside has a helical coil that mixes the sample flowing there through to ensure uniformity of the sample including uniform temperature. Otherwise, the outside portion of the sample will have a tendency to cool down more than the inside portion of the sample. The static mixer 208 makes sure that does not occur. From the static mixer 208, temperature is measured by temperature indicator 210 prior to the sample flowing into the steam heat exchanger 38. From the steam heat exchanger 38, the sample flows through a second static mixer 212, after which the temperature is again measured by temperature indicator 214. Normally, the sample would flow from the static mixer 212 through validation valve 216 out condition supply line 40 to the analyzer 20.

However, if someone wanted to check to see if analyzer 20 was working properly, a validation sample of known characteristics can feed through control valve 218 and through validation valve 216 to conditioned sample line 40. Pressured air is received through pressure line 220 from the analyzer 20 to operate the validation valve 216. A validation sample can be used to determine proper operation of the analyzer 20. The validation sample is received through the validation port 222 into the sample conditioning system 28. Temperature and/or pressure of the validation sample can be measured by temperature/pressure gauge 224.

If the sample conditioning system 28 needs to be flushed, a maintenance flush can be inserted through maintenance flush line 226, control valve 228 and check valve 230 to back flush out the sample supply line 180. If there are particles built up in the “Y” strainer 186, the flush will help remove those particles.

Steam for the steam heat exchanger 38 of the sample conditioning system 28 is provided via the steam inlet line 232 through control valve 234 to a pressure regulator 236. From the pressure regulator 236, the steam feeds through steam coil 238 to the steam heat exchanger 38. The pressure regulator 236 is specifically designed to manage steam and the temperatures of steam. Pressure and temperature can be set according to standard steam tables. The steam coil 238 is used with the pressure regulator 236 to control temperature and pressure of the steam. The steam coil 238 will give off heat to lower the temperature of the steam. By the setting of the pressure regulator 236 and use of the steam coil 238, the temperature of the steam in the steam heat exchanger 38 can be regulated. By use of a steam control needle valve 240, the steam control needle valve 240 allows steam above a particular temperature or pressure to flow there through and out control valve 242 to steam return line 244.

If the pressure of the steam inside of steam coil 238 is too high as determined by pressure gauge 246, relief valve 248 will open, discharging steam out the steam relief line 250.

By use of the sample conditioning system 28 as explained in detail in FIGS. 7 and 8, the temperature of the sample coming in through sample supply line 180 is normally at approximately 240° C. ±20° C. The sample is conditioned and the temperature lowered to approximately 135° C. ±5° C. Before being fed to the analyzer 20 by adjusting the pressure of the steam, the temperature of the steam can likewise be adjusted.

In a typical test for an analyzer 20 with a sample conditioning system 28, measurements can be made for the flow rate, viscosity and temperature as is shown in FIG. 10. Normally the viscosity and temperature would be shown as averages rather than instantaneous measurements.

If the purged enclosure 46 needs to be cooled due to the temperature of the environment in which it operates, FIG. 9 illustrates a vortex cooler inside the purged enclosure 46. Pressurized air connects via fitting 252. Almost every refinery has pressurized air readily available therein. From fitting 252, the pressurized air flows through control solenoid valve 254 in tube 256 to the vortex cooler 258. The vortex cooler 258 discharges hot air through fitting 260 and exhaust muffler 262 to atmosphere. Due to the vortex effect of the vortex cooler 258, cool air will discharge through a cooling tube assembly 264 to the inside of the purged enclosure 46. There are many different manufacturers of vortex coolers with a typical example being sold under the trade name Vortec®.

What I claim is:

1. An apparatus for measuring by an operator density and viscosity of a fluid during refinement in a hostile environment, a source of power and pressurized air being available during said refinement, said apparatus comprising:
A. a non-purged enclosure having:
(1) a bypass line with said fluid flowing there through;
(2) a densitometer connected to said source of power;
(3) a viscometer connected to said source of power;
(4) pneumatic valves in said bypass line for periodically interrupting fluid flowing in said bypass line to send a sample of said fluid to said densitometer, said viscometer subsequently receiving said sample from said densitometer;
(5) a heater connected to said source of power for maintaining temperature in said non-purged enclosure within an acceptable enclosure temperature range;
B. a purged enclosure having:
(1) a computer receiving power from said source of power;
(2) a solenoid operated by said computer for periodically connecting said pressurized air to said pneumatic valves in said non-purged enclosure;
(3) a thermoelectric heater/cooler controlling a heat pipe extending from said purged enclosure to said viscometer in said non-purged enclosure;
C. said computer controlling operation of said solenoid, and receiving data from said densitometer and/or said viscometer to operate said thermoelectric heater/cooler which controls temperature of said sample being measured in said viscometer via said heat pipe.

2. An apparatus for measuring density and viscosity of a fluid during refinement in a hostile environment as recited in claim 1 further comprising a sample conditioning system for first receiving said fluid and flowing said fluid through a heat exchanger to insure said fluid is within a general predetermined temperature range before flowing to said non-purged enclosure.

3. An apparatus for measuring density and viscosity of a fluid during refinement in a hostile environment as recited in claim 2 wherein said sample conditioning system has a booster pump for drawing said fluid from said non-purged enclosure.

4. An apparatus for measuring density and viscosity of a fluid during refinement in a hostile environment as recited in claim 3 wherein said purged enclosure has a purge system for removing explosive gases therefrom and a vortex coder for maintaining temperature therein.

5. An apparatus for measuring density and viscosity of a fluid during refinement in a hostile environment as recited in claim 4 wherein said purged enclosure has external feedback and/or controls from said computer to said operator.

6. An apparatus for measuring density and viscosity of a fluid during refinement in a hostile environment as recited in claim 5 wherein said heat pipe controls measuring temperature of said fluid within a narrow temperature range inside said viscometer.

7. An apparatus for measuring density and viscosity of a fluid during refinement in a hostile environment as recited in claim 6 wherein seal off glands are provided for connections between said non-purged enclosure and said purged enclosure.

8. A method of operating a sample conditioning system for use in a refinery having pressurized air and steam, said sample conditioning system preparing a sample from a fluid such as asphalt during the refinement process, but before testing to determine viscosity and/or density, said method including the following steps:
continuously flowing said fluid through a bypass in said sample conditioning system;
selecting sample flow from a Y strainer in said bypass, said Y strainer removing particles;
first mixing said sample flow in a first static mixer;
cooling said sample flow in a steam heat exchanger to an approximate predetermined temperature;
second mixing of said sample flow in a second static mixer to insure uniformity of said approximate predetermined temperature within said sample flow;
delivering said sample flow to a viscosity analyzer;
returning said sample flow after testing by said viscosity analyzer to said refinement process; and
validating said sample conditioning system by running a validation sample through said viscosity analyzer, said validating step including activation of validation valves by said pressurized air to change from said sample flow to said validation sample.

9. A method of operating a sample conditioning system for use in a refinery as recited in claim 8 including in said returning step a booster pump for creating a vacuum to draw said sample from said viscosity analyzer.

10. A method of operating a sample conditioning system for use in a refinery as recited in claim 9 including an additional step of flushing said sample out of said sample conditioning system through use of said pressurized air.

11. A method of operating a sample conditioning system for use in a refinery as recited in claim 10 including measuring pressure and temperature of said fluid and said sample during the preceding steps.
